(19) United States
(12) Patent Application Publication
(10) Pub. No.: US 2023/0218273 A1
(43) Pub. Date: Jul. 13, 2023

(54) THICKNESS MEASUREMENT DEVICE

(71) Applicant: SEIKO EPSON CORPORATION, TOKYO (JP)

(72) Inventors: Seiji IZUO, Shiojiri-shi (JP); Yoshio ARAI, Shiojiri-shi (JP)

(21) Appl. No.: 18/150,828

(22) Filed: Jan. 6, 2023

(30) Foreign Application Priority Data
Jan. 7, 2022 (JP) 2022-001478

(51) Int. Cl. A61B 8/00

(52) U.S. Cl. CPC A61B 8/4477; A61B 8/4209

(57) ABSTRACT

There is provided a thickness measurement device for measuring a thickness of a measurement target by using an ultrasonic wave, the thickness measurement device being to be attached to an object including the measurement target therein. The thickness measurement device includes a plurality of ultrasonic elements each configured to transmit the ultrasonic wave to the measurement target from a surface of the object, receive a reflected wave reflected by the measurement target, and output a reception signal, and a controller configured to control the ultrasonic elements. The plurality of ultrasonic elements transmit the ultrasonic waves in directions different from one another, and the controller compares a signal intensity of the reception signal with a predetermined threshold, and measures the thickness of the measurement target based on the reception signal having a signal intensity larger than the threshold.

The present application is based on, and claims priority from JP Application Serial Number 2022-001478, filed Jan. 7, 2022, the disclosure of which is hereby incorporated by reference herein in its entirety.

### BACKGROUND

### 1. Technical Field

The present disclosure relates to a thickness measurement device that measures a thickness of a measurement target inside an object.

### 2. Related Art

In the related art, a thickness measurement device that measures a thickness of a measurement target using an ultrasonic wave has been known (see JP-A-S61-220634, JP-A-2015-66219, and JP-A-2003-4430).

For example, a thickness measurement device disclosed in JP-A-S61-220634 transmits an ultrasonic wave from an ultrasonic probe into a body, and the ultrasonic probe receives reflected waves reflected by a boundary surface between a measurement target and a layer adjacent to the measurement target. Then, a distance to the boundary surface is measured based on a time from a transmission timing of the ultrasonic wave to a reception timing of the reflected wave.

However, in the thickness measurement device of the related art as described above, it is necessary to bring a probe into contact with a body surface such that a transmission direction of the ultrasonic wave transmitted from the ultrasonic probe is perpendicular to a surface of the measurement target, and a technique skilled for a measurer is required. For example, in JP-A-S-61-220634, a sebum thickness is measured by measuring a reflected wave on a surface of a muscle tissue, and in this case, it is necessary to operate the probe such that the transmission direction of the ultrasonic wave is perpendicular to the surface of the muscle tissue, and when the transmission direction of the ultrasonic wave is inclined from a normal direction of the surface of the muscle tissue, sound pressure of the reflected wave decreases, resulting in a decrease in measurement accuracy. Further, in JP-A-2003-4430, a holder that holds a golf ball is provided in a container filled with a liquid, and ultrasonic measurement is performed on the golf ball with the liquid interposed by an ultrasonic wave transmitting element provided in the holder. In this configuration, it is necessary to interpose the liquid between the golf ball and the holder in which the ultrasonic wave transmitting element is provided, and thus a configuration of the device is complicated.

### SUMMARY

A thickness measurement device according to a first aspect of the present disclosure is a thickness measurement device for measuring a thickness of a measurement target by using an ultrasonic wave, and the thickness measurement device is to be attached to an object including the measurement target therein. The thickness measurement device includes a plurality of ultrasonic elements each configured to transmit the ultrasonic wave to the measurement target from a surface of the object, receive a reflected wave reflected by the measurement target, and output a reception signal, and a controller configured to control the ultrasonic elements. The plurality of ultrasonic elements transmit the ultrasonic waves in directions different from one another, and the controller compares a signal intensity of the reception signal with a predetermined threshold, and measures the thickness of the measurement target based on the reception signal having a signal intensity larger than the threshold.

### BRIEF DESCRIPTION OF THE DRAWINGS

FIG. 1 is a block diagram showing a schematic configuration of a thickness measurement device according to a first embodiment.

FIG. 2 is a schematic perspective view showing an ultrasonic probe fixed to one surface of a belt according to the first embodiment.

FIG. 3 is a schematic cross-sectional view showing a cross section along an X axis in FIG. 2.

FIG. 4 is a schematic cross-sectional view showing a cross section along a Y axis in FIG. 2.

FIG. 5 is a flowchart showing a thickness measurement method according to the first embodiment.

FIG. 6 is a diagram showing an example of a positional relationship between ultrasonic elements and a measurement target in step S2 of FIG. 5.

FIG. 7 is a diagram showing an example of the positional relationship between the ultrasonic elements and the measurement target in step S2 of FIG. 5.

FIG. 8 is a diagram showing an example of temporal changes in reception signals from the ultrasonic elements according to the first embodiment.

FIG. 9 is a perspective view of an ultrasonic probe in a measurement unit according to a second embodiment.

FIG. 10 is a schematic cross-sectional view showing a cross section along an X axis of the ultrasonic probe of FIG. 9.

FIG. 11 is a diagram showing an example of a positional relationship between ultrasonic elements and a measurement target in step S2.

FIG. 12 is a diagram showing an example of the positional relationship between the ultrasonic elements and the measurement target in step S2.

FIG. 13 is a diagram showing a schematic configuration of an ultrasonic probe according to a second modification.

FIG. 14 is a diagram showing a schematic configuration of another ultrasonic probe according to the second modification.

FIG. 15 is a diagram showing a schematic configuration of still another ultrasonic probe according to the second modification.

### DESCRIPTION OF EXEMPLARY EMBODIMENTS

### First Embodiment

Hereinafter, a thickness measurement device according to a first embodiment will be described.

In the present embodiment, a thickness measurement device that is fixed to a surface of an object and measures a thickness of a measurement target inside the object by detecting a boundary of the measurement target will be described as an example. Examples of the object include various structures such as buildings and living bodies such as human bodies, and examples of the measurement target include reinforcing bars inside the buildings, organs, muscles, fat, bones inside the living bodies, and the like.

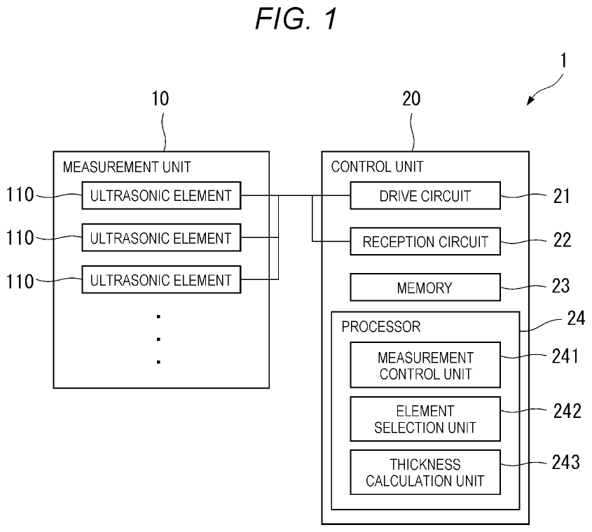

FIG. 1 is a block diagram showing a schematic configuration of a thickness measurement device 1 according to the present embodiment.

As shown in FIG. 1, the thickness measurement device 1 according to the present embodiment includes a measurement unit 10 and a control unit 20.

### Configuration of Measurement Unit 10

The measurement unit 10 is attachable to the object, and performs ultrasonic measurement on an inside of the object.

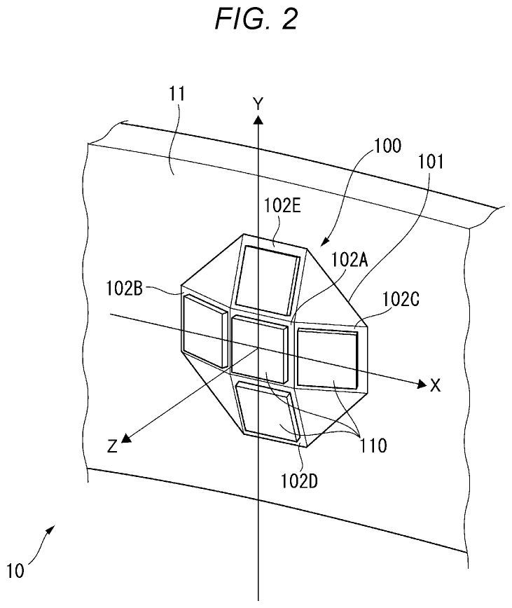

The measurement unit 10 includes, for example, an ultrasonic probe 100 and a fixing unit 11 (see FIGS. 2 to 4) that fixes the ultrasonic probe 100 to the object. A configuration of the fixing unit 11 is not particularly limited, and for example, a configuration in which the fixing unit 11 is attachable to the object and causes the ultrasonic probe 100 to be in close contact with the object in a state of being attached to the object can be exemplified. In addition, as the fixing unit 11, a structure in which the ultrasonic probe 100 is provided is exemplified, and the fixing unit 11 is not limited thereto as long as the ultrasonic probe 100 is fixed to the object. For example, a viscous gel such as gel may be used as a fixing unit to bond and fix the ultrasonic probe 100 and the object.

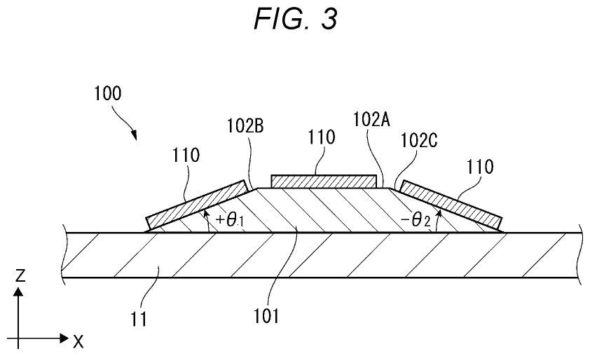

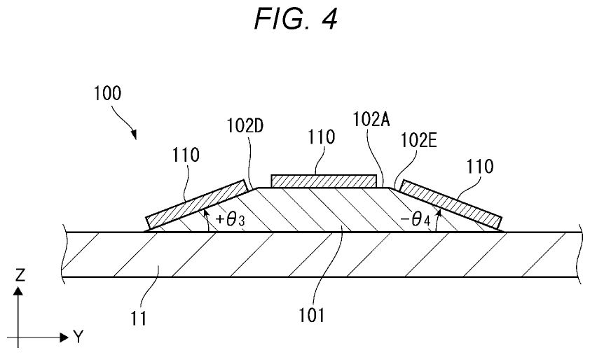

FIG. 2 is a schematic perspective view showing the ultrasonic probe 100 provided at the fixing unit 11. FIG. 3 is a schematic cross-sectional view showing a cross section along an X axis of FIG. 2, and FIG. 4 is a schematic cross-sectional view showing a cross section along a Y axis of FIG. 2. Here, the X axis is an axis parallel to one direction along the surface of the object when the ultrasonic probe 100 is fixed to the object, and the Y axis is an axis along the surface of the object and orthogonal to the X axis. Further, an axis orthogonal to the X axis and the Y axis is taken as a Z axis. A +Z side in the Z axis is a direction from the fixing unit 11 toward the object.

As shown in FIGS. 2 to 4, the ultrasonic probe 100 according to the present embodiment includes a holder 101 and a plurality of ultrasonic elements 110.

The holder 101 is a member that holds the ultrasonic elements 110, and is fixed to the fixing unit 11 in the present embodiment. In the present embodiment, the holder 101 has a plurality of holding surfaces 102A to 102E for holding the ultrasonic elements 110, and normal directions of the holding surfaces 102A to 102E are different from one another.

For example, in the present embodiment, the first holding surface 102A is parallel to an XY plane, and the normal direction thereof is parallel to the Z axis.

The second holding surface 102B is disposed on a −X side of the first holding surface 102A. The second holding surface 102B is parallel to the Y axis and is inclined at an angle of +θ₁ around the Y axis with respect to the XY plane. Therefore, the normal direction of the second holding surface 102B is inclined at an angle of θ₁ from a Z direction to the −X side.

The third holding surface 102C is disposed on a +X side of the first holding surface 102A. The third holding surface 102C is parallel to the Y axis and is inclined at an angle of −θ₂ around the Y axis with respect to the XY plane. Therefore, the normal direction of the third holding surface 102C is inclined at an angle of θ₂ from the Z direction to the +X side.

The fourth holding surface 102D is disposed on a −Y side of the first holding surface 102A. As shown in FIG. 4, the fourth holding surface 102D is parallel to the X axis, and is inclined at an angle of +θ₃ around the X axis with respect to the XY plane. Therefore, the normal direction of the fourth holding surface 102D is inclined at an angle of θ₃ from the Z direction to the −Y side.

The fifth holding surface 102E is disposed on a +Y side of the first holding surface 102A. The fifth holding surface 102E is parallel to the Y axis and is inclined at an angle of −θ₄ around the X axis with respect to the XY plane. Therefore, the normal direction of the fifth holding surface 102E is inclined at an angle of θ₄ from the Z direction to the +Y side.

All of the angles θ₁, θ₂, θ₃, and θ₄ may be the same, some of them may be different, or all of them may be different.

The plurality of ultrasonic elements 110 transmit ultrasonic waves toward the object and receive reflected waves reflected by the measurement target inside the object. The ultrasonic element 110 is not particularly limited as long as it is an element capable of transmitting and receiving the ultrasonic waves.

For example, a thin-film ultrasonic element may be used in which a plurality of ultrasonic transducers in which piezoelectric elements are disposed at thin-film vibrating units are disposed in an array, and the ultrasonic waves are transmitted by vibrating the vibrating units by applying voltages to the piezoelectric elements. In such a thin-film ultrasonic element, a reception signal is output from the piezoelectric elements by vibrating a vibration film by the reflected wave.

Alternatively, a bulk-type ultrasonic element may be used in which the ultrasonic wave is transmitted by vibrating a piezoelectric body itself by applying a voltage to the piezoelectric body, and the reflected wave is detected by the reception signal output by distortion of the piezoelectric body itself due to the reflected wave.

In order to reduce a thickness and a size of the measurement unit 10, it is preferable to use the thin-film ultrasonic element.

The plurality of ultrasonic elements 110 are provided at the holding surfaces 102A to 102E, respectively.

Therefore, the ultrasonic element 110 provided at the first holding surface 102A transmits the ultrasonic wave toward the object (to the +Z side) along the Z direction. The ultrasonic element 110 provided at the second holding surface 102B transmits the ultrasonic wave in a direction inclined at an angle of θ₁ to the −X side from the Z direction. The ultrasonic element 110 provided at the third holding surface 102C transmits the ultrasonic wave in a direction inclined at an angle of θ₂ to the +X side from the Z direction. That is, the ultrasonic elements 110 disposed along the X axis transmit the ultrasonic waves in directions away from one another in an XZ plane (first plane).

The ultrasonic element 110 provided at the fourth holding surface 102D transmits the ultrasonic wave in a direction inclined at an angle of θ₃ to the −Y side from the Z direction. The ultrasonic element 110 provided at the fifth holding surface 102E transmits the ultrasonic wave in a direction inclined at an angle of θ₄ to the +Y side from the Z direction. That is, the ultrasonic elements 110 disposed along the Y axis transmit the ultrasonic waves in directions away from one another in a YZ plane (second plane).

### Configuration of Control Unit 20

For example, the control unit 20 may be provided at a surface of the fixing unit 11 of the measurement unit 10 opposite to a surface at which the ultrasonic elements 110 are provided, or may be provided separately from the measurement unit 10 and capable of communicating with the measurement unit 10 in a wired or wireless manner.

The control unit 20 corresponds to a controller according to the present disclosure, controls an operation of each of the ultrasonic elements 110, and measures the thickness of the measurement target inside the object based on the reception signals obtained from the ultrasonic elements 110.

Specifically, as shown in FIG. 1, the control unit 20 includes a drive circuit 21 that drives the ultrasonic elements 110, a reception circuit 22 that processes the reception signals, a memory 23 that stores various types of information, and one or a plurality of processors 24.

Based on a command from the processor 24, the drive circuit 21 outputs a drive signal to each of the ultrasonic elements 110 to drive the ultrasonic elements 110, and causes the ultrasonic elements 110 to transmit the ultrasonic waves. The drive circuit 21 may be provided for each of the ultrasonic elements 110, and one drive circuit 21 and the plurality of ultrasonic elements 110 may be coupled by a switch circuit such that the drive circuit 21 that outputs the drive signal can be selected by the switch circuit.

The reception circuit 22 processes the reception signals output from the ultrasonic elements 110, and outputs the processed reception signals to the processor 24. The reception circuit 22 may be provided for each of the ultrasonic elements 110, and one reception circuit 22 and the plurality of ultrasonic elements 110 may be coupled via a switch circuit.

The memory 23 stores various programs including a measurement program for measuring the thickness of the measurement target by transmission and reception processing on the ultrasonic waves, and various pieces of data used in the various programs.

The processor 24 reads and executes the various programs stored in the memory 23 to perform various types of calculation processing. Specifically, the processor 24 functions as a measurement control unit 241, an element selection unit 242, and a thickness calculation unit 243 by executing the various programs. The measurement control unit 241 outputs a transmission command of the ultrasonic wave to the drive circuit 21, causes the ultrasonic element 110 to transmit the ultrasonic wave, and acquires the reception signal received from the reception circuit 22. At this time, the measurement control unit 241 sequentially drives the plurality of ultrasonic elements 110 independently, and acquires the reception signal from the ultrasonic elements 110.

The element selection unit 242 compares the reception signals obtained from the ultrasonic elements 110, and selects an ultrasonic element 110 corresponding to a reception signal having a signal intensity equal to or greater than a predetermined threshold as the ultrasonic element 110 for measuring the thickness of the measurement target.

The thickness calculation unit 243 calculates the thickness of the measurement target based on the reception signal obtained from the ultrasonic element 110 selected by the element selection unit 242 and a transmission timing of the ultrasonic wave to the ultrasonic element 110.

### Thickness Measurement Method

Next, a thickness measurement method according to the present embodiment will be described.

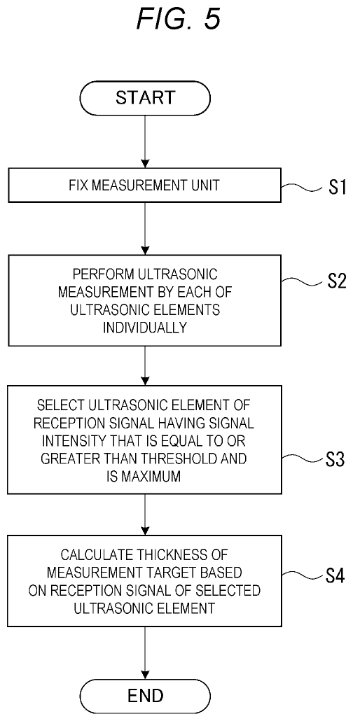

FIG. 5 is a flowchart of the thickness measurement method according to the present embodiment.

When the thickness of the measurement target inside the object is measured by the thickness measurement device 1 according to the present embodiment, first, a user fixes the measurement unit 10 to the object to be measured using the fixing unit 11, and brings the ultrasonic probe 100 into close contact with the object (step S1).

Thereafter, the measurement control unit 241 sequentially drives the plurality of ultrasonic elements 110 to transmit the ultrasonic waves, causes the ultrasonic elements 110 to receive the ultrasonic waves reflected from the inside of the object, and measures the reception signals (step S2). That is, the ultrasonic measurement including transmission processing of the ultrasonic waves and reception processing of the reflected waves by the ultrasonic elements 110 is individually performed by each of the ultrasonic elements 110.

Then, the element selection unit 242 specifies a reception signal corresponding to a reception signal having a signal intensity that is equal to or greater than a predetermined threshold and is maximum from the obtained reception signals from the ultrasonic elements 110, and specifies the ultrasonic element 110 that outputs the reception signal as the ultrasonic element 110 for measurement (step S3).

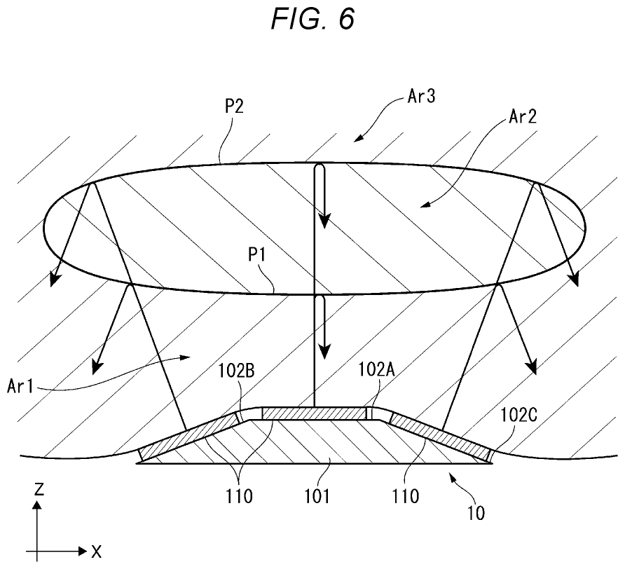

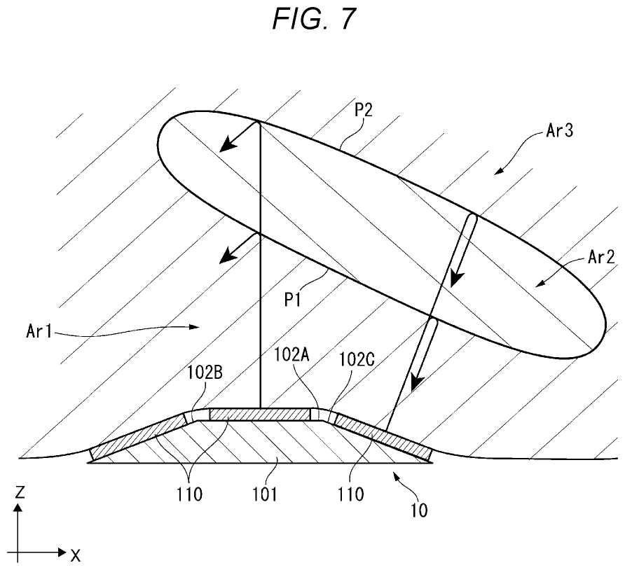

FIGS. 6 and 7 are diagrams showing examples of a positional relationship between the ultrasonic elements 110 and a muscle tissue in step S2 of FIG. 5. In the present embodiment, a second portion Ar2 inside the object is the measurement target. A portion between the surface of the object and the second portion Ar2 is referred to as a first portion Ar1, and in the present embodiment, the first portion Ar1 is disposed at a position closest to the surface of the object. A portion that is farther from the surface of the object than the second portion Ar2 and is adjacent to the second portion Ar2 is referred to as a third portion Ar3.

For example, in the example shown in FIG. 6, the ultrasonic element 110 provided at the first holding surface 102A transmits the ultrasonic wave substantially perpendicular to a surface of the second portion Ar2 which is the measurement target. Meanwhile, the ultrasonic waves transmitted from the ultrasonic elements 110 of the other holding surfaces 102B to 102E are transmitted more obliquely with respect to a normal line of the surface of the second portion Ar2 than the ultrasonic element 110 of the first holding surface 102A. In FIGS. 6 and 7, illustration of the ultrasonic elements 110 held at the holding surfaces 102D and 102E is omitted.

In this case, the ultrasonic wave transmitted from the ultrasonic element 110 of the first holding surface 102A is substantially regularly reflected by the surface of the second portion Ar2, and a reflected wave having relatively strong sound pressure is received by the ultrasonic element 110. Therefore, a reception signal having a large signal intensity is output from the ultrasonic element 110 of the first holding surface 102A. In the ultrasonic elements 110 of the other holding surfaces 102B to 102E, the sound pressure of the received reflected waves is smaller and the signal intensities of the reception signals are less than that of the ultrasonic element 110 of the first holding surface 102A.

In the example shown in FIG. 7, the ultrasonic elements 110 provided at the third holding surface 102C transmit the ultrasonic wave substantially perpendicularly to the surface of the second portion Ar2, and the ultrasonic elements 110 of the other holding surfaces 102A, 102B, 102D, and 102E transmit the ultrasonic waves at angles inclined with respect to a normal line of a surface of the muscle. Therefore, in this case, a reception signal having a signal intensity larger than that of the other ultrasonic elements 110 is output from the ultrasonic element 110 of the third holding surface 102C.

When the thickness of the measurement target inside the object is measured, an ultrasonic wave having a frequency capable of reaching the first portion Ar1, the second portion Ar2, and the third portion Ar3 is used. Accordingly, a part of the ultrasonic wave is reflected at a boundary (a first boundary P1) between the first portion Ar1 and the second portion Ar2, and a part of the ultrasonic wave passing through the first boundary P1 is reflected at a boundary (a second boundary P2) between the second portion Ar2 and the third portion Ar3.

Therefore, each ultrasonic element 110 continues the reception processing on the ultrasonic wave for a predetermined period from the transmission timing of the ultrasonic wave such that the reception signal by the reflected wave reflected at the first boundary P1 and the reception signal by the reflected wave reflected at the second boundary P2 are obtained.

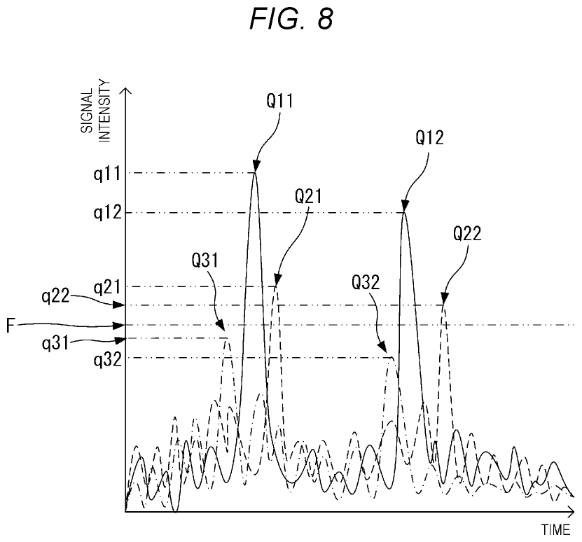

FIG. 8 is a diagram showing an example of temporal changes in the reception signals from the ultrasonic elements 110. A solid line indicates the reception signal (a first reception signal) output from the ultrasonic element 110 of the first holding surface 102A, a broken line indicates the reception signal (a second reception signal) output from the ultrasonic element 110 of the second holding surface 102B, and a one-dot chain line indicates the reception signal (a third reception signal) output from the ultrasonic element 110 of the third holding surface 102C.

As shown in FIG. 8, each of the reception signals has a first peak value corresponding to the first boundary P1 and a second peak value corresponding to the second boundary P2.

In FIG. 8, a position of the first peak value of the first reception signal is indicated by Q11, and a position of the second peak value is indicated by Q12. A position of the first peak value of the second reception signal is indicated by Q21, and a position of the second peak value is indicated by Q22. A position of the first peak value of the third reception signal is indicated by Q31, and a position of the second peak value is indicated by Q32. The first peak value and the second peak value of the first reception signal are indicated by q11 and q12, respectively, the first peak value and the second peak value of the second reception signal are indicated by q21 and q22, respectively, and the first peak value and the second peak value of the third reception signal are indicated by q31 and q32, respectively.

In the present embodiment, in step S3, the element selection unit 242 first specifies a reception signal in which both the first peak value and the second peak value are equal to or greater than a threshold F.

For example, in the example of FIG. 8, the first peak value q31 and the second peak value q32 of the third reception signal output from the ultrasonic element 110 of the third holding surface 102C are less than the threshold F. Therefore, in step S3, the ultrasonic element 110 of the third holding surface 102C is excluded from the elements for measurement.

On the other hand, since both the first peak values q11 and q21 and the second peak values q12 and q22 of the reception signals output from the ultrasonic elements 110 of the first holding surface 102A and the second holding surface 102B exceed the threshold F, the ultrasonic elements 110 of the first holding surface 102A and the second holding surface 102B are specified as candidates of the element for measurement. Further, the first peak value q11 and the second peak value q12 of the first reception signal from the ultrasonic element 110 of the first holding surface 102A are larger than the first peak value q21 and the second peak value q22 of the second reception signal from the ultrasonic element 110 of the second holding surface 102B. Therefore, in the example of FIG. 8, the ultrasonic element 110 of the first holding surface 102A is selected as the element for measurement.

When there are a plurality of reception signals in which both the first peak value and the second peak value exceed the threshold, and a reception signal having a maximum first peak value and a reception signal having a maximum second peak value are different from each other, the element for measurement may be selected based on a ratio of the peak values. That is, a reception signal whose peak value that is not maximum is close to a maximum value is used for the measurement. For example, when the first peak value q11 of the first reception signal and the first peak value q21 of the second reception signal satisfy q11>q21 and the second peak value q12 of the first reception signal and the second peak value q22 of the second reception signal satisfy q22>q12, q21/q11 is compared with q12/q22. When q21/q11>q12/q22, the ultrasonic element 110 that outputs the second reception signal is selected as the element for measurement, and when q21/q11<q12/q22, the ultrasonic element 110 that outputs the first reception signal is selected as the element for measurement.

Alternatively, an ultrasonic element 110 that outputs a reception signal having a maximum sum of the first peak value and the second peak value may be selected as the element for measurement.

After step S3, the thickness calculation unit 243 calculates a thickness of the second portion Ar2 which is the measurement target based on the reception signal output from the ultrasonic element 110 selected in step S3 and an output timing of the transmission command to the ultrasonic element 110 (step S4).

That is, the thickness calculation unit 243 can calculate a distance L1 (a thickness of the first portion Ar1) from the ultrasonic element 110 to the first boundary P1 during a time (a first time) from the transmission timing of the ultrasonic wave until the first peak value of the reception signal is obtained. Similarly, a distance L2 from the ultrasonic element 110 to the second boundary P2 can be calculated during a time (a second time) from the transmission timing until the second peak value of the reception signal is obtained. Accordingly, the thickness L of the second portion Ar2 can be calculated by L=L2−L1.

### Functions and Effects of Present Embodiment

The thickness measurement device 1 according to the first embodiment includes the measurement unit 10 and the control unit 20 (controller). The measurement unit 10 includes the plurality of ultrasonic elements 110 that transmit the ultrasonic waves from the surface of the object to the inside thereof, receive the reflected waves reflected by the surface of the second portion Ar2 which is the measurement target inside the object, and output the reception signals, and the plurality of ultrasonic elements 110 transmit the ultrasonic waves in directions different from one another. The control unit 20 compares the signal intensity of the reception signal with the predetermined threshold F, and measures the thickness of the second portion Ar2 based on the reception signal having a signal intensity larger than the threshold F.

When the signal intensity of the reception signal exceeds the threshold F, it can be determined that the ultrasonic wave is incident on the surface (the first boundary P1 or the second boundary P2) of the second portion Ar2 at an angle close to a right angle, and the reflected wave having high sound pressure is received by the ultrasonic element 110. Therefore, by measuring the thickness of the second portion Ar2 based on such a reception signal, an appropriate thickness substantially along a normal direction of the surface of the second portion Ar2 can be measured.

When the ultrasonic wave is input obliquely with respect to the normal line of the surface of the second portion Ar2, the ultrasonic wave having a specular reflection component directed from the surface of the second portion Ar2 toward the ultrasonic element 110 decreases. That is, the sound pressure of the ultrasonic wave is reduced, and a possibility that the reception signal is buried in noise is increased, resulting in a decrease in the measurement accuracy. In contrast, in the present embodiment, since the signal intensity of the reception signal exceeds the threshold F, the reception signal is less likely to be buried in the noise, and the measurement accuracy can be improved.

In the present embodiment, the plurality of ultrasonic elements 110 transmit the ultrasonic waves in directions away from one another. Specifically, the ultrasonic probe 100 includes the plurality of ultrasonic elements 110 disposed along the X axis and the plurality of ultrasonic elements 110 disposed along the Y axis. The plurality of ultrasonic elements 110 disposed along the X axis transmit the ultrasonic waves in directions away from one another in the XZ plane, and the plurality of ultrasonic elements 110 disposed along the Y axis transmit the ultrasonic waves in directions away from one another in the YZ plane. Accordingly, the ultrasonic waves can be transmitted over a wide range in a three-dimensional space, the ultrasonic element 110 capable of transmitting the ultrasonic wave substantially perpendicularly to the surface of the second portion Ar2 can be easily specified without changing an attachment position of the measurement unit 10 with respect to the object, and the measurement accuracy can be improved by calculating the thickness of the second portion Ar2 based on an appropriate reception signal from the ultrasonic element 110.

### Second Embodiment

The first embodiment discloses an example in which the plurality of ultrasonic elements 110 are disposed such that ultrasonic waves are transmitted in directions away from one another. On the other hand, a second embodiment is different from the first embodiment in that the ultrasonic waves transmitted from the plurality of ultrasonic elements are transmitted in directions approaching one another.

In the following description, described items are denoted by the same reference numerals, and the description thereof is omitted or simplified.

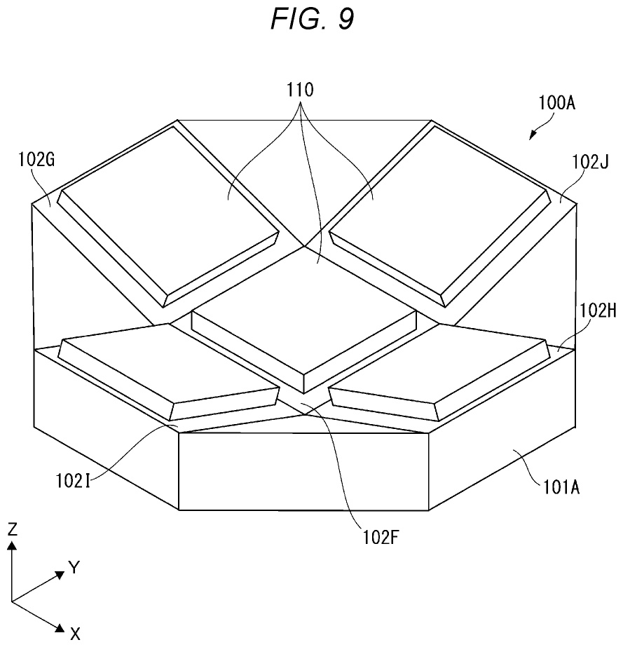

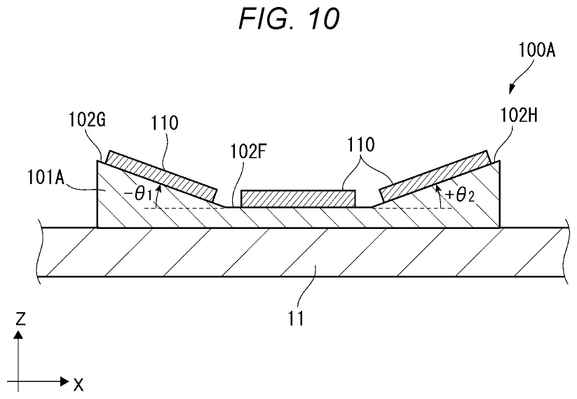

FIG. 9 is a perspective view of an ultrasonic probe 100A in a measurement unit according to the second embodiment, and FIG. 10 is a schematic cross-sectional view showing a cross section along an X axis of the ultrasonic probe 100A of FIG. 9.

As in the first embodiment, the measurement unit according to the present embodiment includes one or more ultrasound probes 100A held by the fixing unit 11.

The ultrasonic probe 100A includes a holder 101A and a plurality of ultrasonic elements 110, and the holder 101A has a plurality of holding surfaces 102F to 102J. In the first embodiment, the holding surfaces 102B to 102E are disposed such that the holder 101 has a projecting shape in which the first holding surface 102A protrudes to the +Z side, and in the second embodiment, the holder 101A is formed in a recessed shape.

That is, in the present embodiment, a seventh holding surface 102G on a −X side of a sixth holding surface 102F parallel to the XY plane is parallel to the Y axis and is inclined at the angle of −θ₁ around the Y axis with respect to the XY plane. An eighth holding surface 102H on a +X side of the sixth holding surface 102F is parallel to the Y axis and is inclined at the angle of +θ₂ around the Y axis with respect to the XY plane.

Although a cross-sectional view of the ultrasonic probe 100A taken along a YZ plane is omitted, a ninth holding surface 102I, the sixth holding surface 102F, and a tenth holding surface 102J that are disposed along the Y axis are also implemented in the same manner. That is, the ninth holding surface 102I on a −Y side of the sixth holding surface 102F is parallel to the X axis and is inclined at the angle of −θ₃ around the X axis with respect to the XY plane. The tenth holding surface 102J on a +Y side of the sixth holding surface 102F is parallel to the X axis and is inclined at the angle of +θ₄ around the X axis with respect to the XY plane.

In the present embodiment, similarly to the first embodiment, the thickness of the second portion Ar2, which is a measurement target inside an object, can be measured by the thickness measurement method shown in FIG. 5.

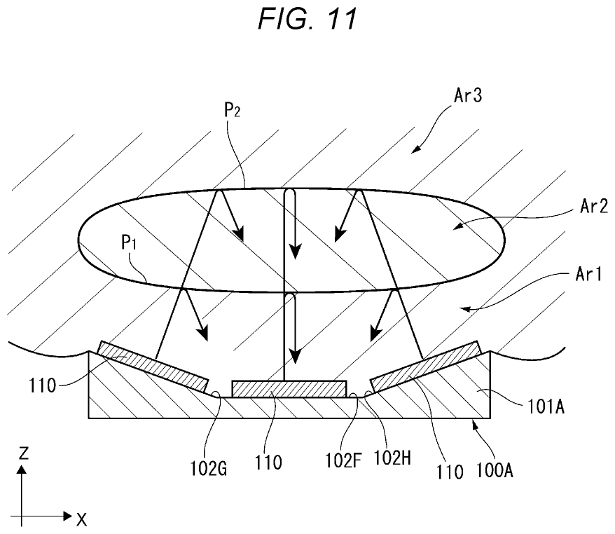

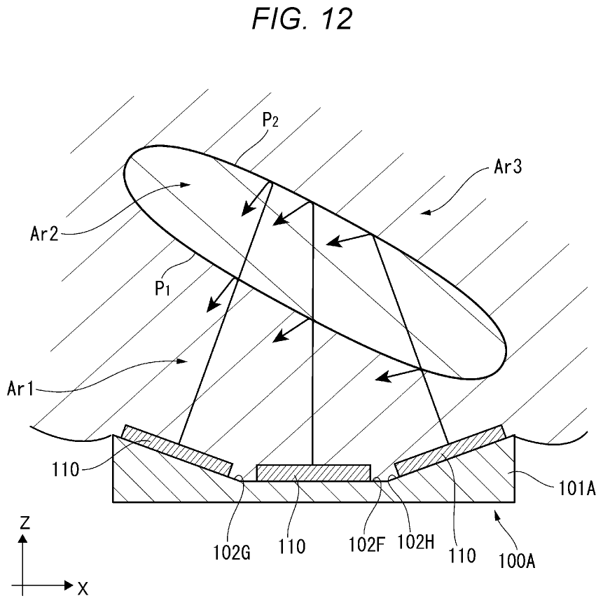

FIGS. 11 and 12 are diagrams showing examples of a positional relationship between the ultrasonic elements 110 and the second portion Ar2 in step S2.

In the present embodiment, when the measurement unit is attached to the object and ultrasonic measurement processing of step S2 is performed, as shown in FIGS. 11 and 12, the ultrasonic waves are transmitted from the ultrasonic elements 110 in directions approaching one another.

For example, in the example shown in FIG. 11, the ultrasonic element 110 provided at the sixth holding surface 102F transmits the ultrasonic wave substantially perpendicular to the surface of the second portion Ar2, and the other ultrasonic elements 110 transmit the ultrasonic waves at an angle inclined with respect to the normal line of the surface of the second portion Ar2. Therefore, the ultrasonic wave transmitted from the ultrasonic element 110 of the sixth holding surface 102F outputs a reception signal having a relatively large signal intensity as compared to the other ultrasonic elements 110.

Accordingly, in step S3, the ultrasonic element 110 provided at the first holding surface 102A is selected as an element for measurement, and in step S4, the thickness of the second portion Ar2 is calculated based on the reception signal of the ultrasonic element 110.

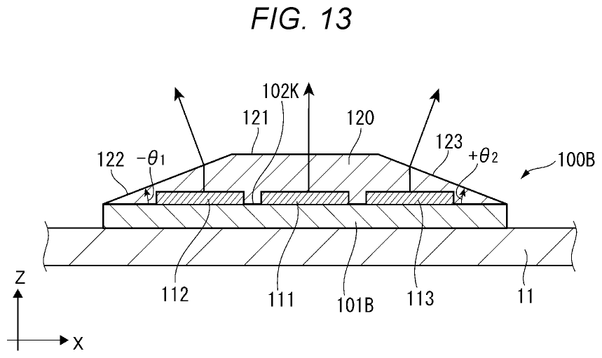

In an example shown in FIG. 13, the ultrasonic element 110 provided at the seventh holding surface 102G transmits the ultrasonic wave substantially perpendicularly to the surface of the second portion Ar2, and the other ultrasonic elements 110 transmit the ultrasonic waves at an angle inclined with respect to the normal line of the surface of the second portion Ar2. Therefore, since the reception signal having a signal intensity larger than that of the other ultrasonic elements 110 is output from the ultrasonic element 110 of the seventh holding surface 102G, the ultrasonic element 110 provided at the seventh holding surface 102G is selected as the element for measurement in step S3.

In the present embodiment, similarly to the first embodiment, the ultrasonic elements 110 of the measurement unit 10 transmit the ultrasonic waves in directions different from one another. Then, the control unit 20 compares the signal intensity of the reception signal with the predetermined threshold F, and the thickness of the second portion Ar2 which is the measurement target is measured based on the reception signal having the signal intensity larger than the threshold F.

Accordingly, similarly to the first embodiment, an appropriate thickness substantially along the normal direction of the surface of the second portion Ar2 can be measured, the reception signal is less likely to be buried in noise, and measurement accuracy is improved.

Further, in the present embodiment, the plurality of ultrasonic elements transmit the ultrasonic waves in directions approaching one another. That is, the ultrasonic elements 110 disposed along the X axis transmit the ultrasonic waves in directions approaching one another in the XZ plane, and the ultrasonic elements 110 disposed along the Y axis transmit the ultrasonic waves in directions approaching one another in the YZ plane. In this case, even when a size of the second portion Ar2 inside the object is small, the thickness of the second portion Ar2 can be suitably measured. For example, suitable measurement can be performed on minute pores or the like present inside concrete of a building.

### Modification

The present disclosure is not limited to the embodiments described above, and configurations obtained through modifications, alterations, and appropriate combinations of the embodiments within a scope of being capable of achieving the object of the present disclosure are included in the present disclosure.

### First Modification

For example, in the first embodiment described above, the element selection unit 242 selects, from among the plurality of ultrasonic elements 110, the ultrasonic element 110 that outputs a reception signal having a signal intensity equal to or greater than a threshold and being maximum.

In contrast, the element selection unit 242 may select a plurality of ultrasonic elements 110 each outputting a reception signal having a signal intensity equal to or greater than the threshold F.

When a maximum first peak value is detected by the ultrasonic element 110 disposed at any one of the holding surfaces 102A to 102E, and a maximum second peak value is detected by the ultrasonic element 110 disposed on any other one of the holding surfaces 102A to 102E, in the first embodiment, an element for measurement is selected based on a ratio of the signal intensity. In contrast, the ultrasonic element 110 (first ultrasonic element) that outputs the reception signal having the maximum first peak value and the ultrasonic element 110 (second ultrasonic element) that outputs the reception signal having the maximum second peak value may be selected as the element for measurement.

As described above, when the plurality of ultrasonic elements 110 are selected, an average value of a muscle thickness calculated by the thickness calculation unit 243 based on the reception signals may be used.

For example, the thickness calculation unit 243 detects, based on the reception signal output from the first ultrasonic element, a first time from a transmission timing of the ultrasonic wave until a first peak value is obtained and a second time from the transmission timing of the ultrasonic wave until a second peak value is obtained, and calculates a thickness of a first measurement target. Similarly, the thickness calculation unit 243 calculates a thickness of a second measurement target based on a first time and a second time based on the reception signal output from the second ultrasonic element. Then, an average value of the thickness of the first measurement target and the thickness of the second measurement target is used as a thickness of a measurement target.

Alternatively, the thickness calculation unit 243 may calculate the muscle thickness based on an average time for which each peak value is obtained. For example, the thickness calculation unit 243 calculates a distance L1′ from the ultrasonic probe 100 to the first boundary P1 based on an average value of the first time until the first peak value of each of the reception signals of the plurality of ultrasonic elements 110 selected as the element for measurement is obtained. A distance L2′ from the ultrasonic probe 100 to the second boundary P2 is calculated based on an average value of the second time until the second peak value of each of the reception signal of the plurality of ultrasonic elements 110 selected as the elements for measurement is obtained. Then, the thickness calculation unit 243 calculates the thickness L of the measurement target by L=L2′−L1′.

Further, the above example is an example of calculating the muscle thickness using an average of the thickness of the measurement target based on the reception signals, or an average of the first time and an average of the second time in the reception signals, and the muscle thickness may be calculated by using other representative values. For example, when there are three or more reception signals having peak values exceeding the threshold, the thickness of the measurement target may be calculated based on each of the reception signals, and the final thickness of the measurement target may be obtained based on a median value or a mode value thereof.

### Second Modification

In the first embodiment, an example in which the ultrasonic waves output from the ultrasonic elements 110 are transmitted in directions away from one another due to the holding surfaces 102A to 102E of the holder 101 has been described. In addition, in the second embodiment, an example in which the ultrasonic waves output from the ultrasonic elements 110 are transmitted in directions approaching one another due to the holding surfaces 102F to 102J of the holder 101A has been described. In contrast, a transmission direction of the ultrasonic wave may be changed by an acoustic lens.

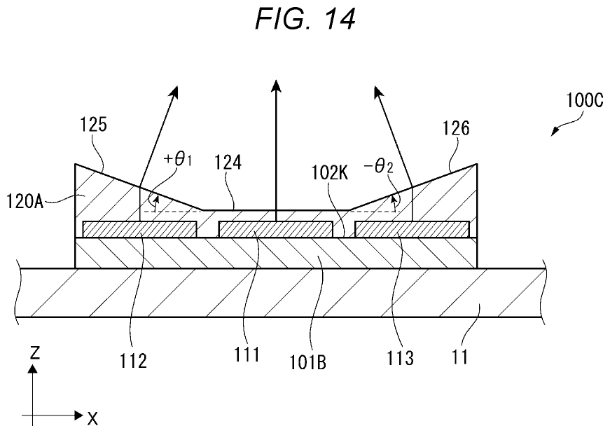

FIGS. 13 and 14 are cross-sectional views showing schematic configurations of ultrasonic probes 100B and 100C according to a second modification.

In the ultrasonic probe 100B of FIG. 13, a plurality of ultrasonic elements (a first ultrasonic element 111, a second ultrasonic element 112, and a third ultrasonic element 113) are disposed along an X direction at a holder 101B having a holding surface 102K parallel to the XY plane. The second ultrasonic element 112 is disposed on a −X side of the first ultrasonic element 111, and the third ultrasonic element 113 is disposed on a +X side of the first ultrasonic element 111.

The acoustic lens 120 is provided to cover the holder 101B and the plurality of ultrasonic elements. The acoustic lens 120 has a first lens surface 121 facing the first ultrasonic element 111, a second lens surface 122 facing the second ultrasonic element 112, and a third lens surface 123 facing the third ultrasonic element 113, and a surface thereof on a +Z side is formed in a projecting shape. That is, the first lens surface 121 is a plane parallel to the XY plane. The second lens surface 122 is parallel to the Y axis and is inclined at the angle of −θ₁ around the Y axis with respect to the XY plane, and the third lens surface 123 is parallel to the Y axis and is inclined at the angle of +θ₂ around the Y axis with respect to the XY plane.

In such a configuration, since the ultrasonic waves are refracted by the acoustic lens 120, the ultrasonic waves transmitted from the ultrasonic elements 111, 112, and 113 can be transmitted in the directions away from one another as in the first embodiment.

Further, in the ultrasonic probe 100C shown in FIG. 14, similarly to the ultrasonic probe 100B, the plurality of ultrasonic elements (the first ultrasonic element 111, the second ultrasonic element 112, and the third ultrasonic element 113) are disposed along the X direction at the holder 101B having the holding surface 102K parallel to the XY plane, and the ultrasonic probe 100C is provided with an acoustic lens 120A covering the holder 101B and the ultrasonic elements 111, 112, and 113.

In the ultrasonic probe 100C, a surface of the acoustic lens 120A on the +Z side is formed in a recessed shape. That is, a fourth lens surface 124 is a plane parallel to the XY plane. A fifth lens surface 125 is parallel to the Y axis and is inclined at the angle of +θ₁ around the Y axis with respect to the XY plane, and a sixth lens surface 126 is parallel to the Y axis and is inclined at the angle of −θ₂ around the Y axis with respect to the XY plane.

In such a configuration, since the ultrasonic waves are refracted by the acoustic lens 120, the ultrasonic waves transmitted from the ultrasonic elements 111, 112, and 113 can be transmitted in the directions approaching one another as in the second embodiment.

The same applies to a case where the ultrasonic elements are disposed in a Y direction.

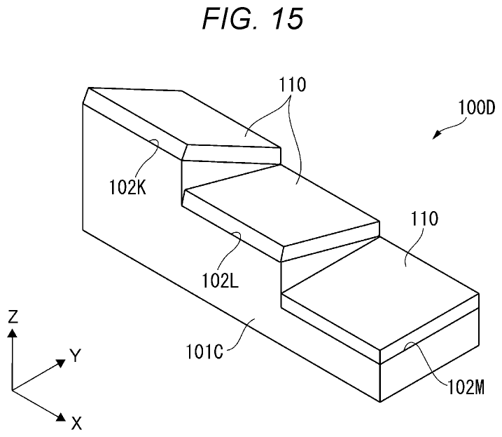

FIG. 15 is a perspective view showing a configuration example of another ultrasonic probe 100D according to the second modification. In the thickness measurement device 1 according to the present disclosure, the ultrasonic elements 110 are disposed as long as the transmission directions of the ultrasonic waves are different from one another. The first embodiment and the second embodiment are examples in which the ultrasonic elements 110 disposed in the X direction transmit the ultrasonic waves that are orthogonal to the Y axis and have different inclination angles with respect to the X axis and the Z axis. In contrast, in the ultrasonic probe 100D of FIG. 15, holding surfaces 102K, 102L, and 102M are provided in a holder 101C, and the holding surfaces 102K, 102L, and 102M are parallel to the X axis and have different inclination angles around the X axis with respect to the XY plane. The holding surfaces 102K, 102L, and 102M are provided with the ultrasonic elements 110, respectively. With such a configuration, the ultrasonic elements 110 can transmit the ultrasonic waves that are orthogonal to the X axis and have different inclination angles with respect to the Y axis and the Z axis.

In such an ultrasonic probe 100D, the ultrasonic waves can be transmitted to the second portion Ar2 having a different depth inside an object. That is, an error due to a difference in a thickness of the first portion Ar1 can be reduced.

### Third Modification

In the above-described embodiments, a thickness of the second portion Ar2 is measured based on reflected waves by the first boundary P1 and the second boundary P2 of the second portion Ar2 which is a measurement target, and a thickness of the first portion Ar1 may be measured based on the reflected waves reflected by the first boundary P1 with the measurement target being the first portion Ar1.

### Overview of Present Disclosure

A thickness measurement device according to a first aspect of the present disclosure is a thickness measurement device for measuring a thickness of a measurement target by using an ultrasonic wave, and the thickness measurement device is to be attached to an object including the measurement target therein. The thickness measurement device includes a plurality of ultrasonic elements each configured to transmit the ultrasonic wave to the measurement target from a surface of the object, receive a reflected wave reflected by the measurement target, and output a reception signal, and a controller configured to control the ultrasonic elements. The plurality of ultrasonic elements transmit the ultrasonic waves in directions different from one another, and the controller compares a signal intensity of the reception signal with a predetermined threshold, and measures the thickness of the measurement target based on the reception signal having a signal intensity larger than the threshold.

Accordingly, the thickness of the measurement target is measured based on the ultrasonic wave incident on a surface of the measurement target at an angle close to a right angle. Therefore, an appropriate thickness substantially along a normal direction of the surface of the measurement target can be measured, and a decrease in measurement accuracy and an increase in a measurement error due to attenuation of the ultrasonic wave can be prevented.

In the thickness measurement device according to the first aspect, the plurality of ultrasonic elements transmit the ultrasonic waves in the directions away from one another.

Accordingly, since the ultrasonic waves can be transmitted in a wide range, the ultrasonic element capable of transmitting the ultrasonic wave substantially perpendicularly to the surface of the measurement target can be easily specified without changing an attachment position of the plurality of ultrasonic elements with respect to the object.

In the thickness measurement device according to the first aspect, the plurality of ultrasonic elements include a plurality of ultrasonic elements disposed along a first axis and a plurality of ultrasonic elements disposed along a second axis orthogonal to the first axis, with an axis orthogonal to the first axis and the second axis as a third axis, the plurality of ultrasonic elements disposed along the first axis transmit the ultrasonic waves in the directions away from one another in a first plane including the first axis and the third axis, and the plurality of ultrasonic elements disposed along the second axis transmit the ultrasonic waves in the directions away from one another in a second plane including the second axis and the third axis.

Accordingly, the ultrasonic waves can be transmitted not only in one plane but also in a wide range of a three-dimensional space, the ultrasonic element capable of appropriately transmitting the ultrasonic wave to the measurement target can be specified, and the measurement accuracy in the thickness measurement can be improved.

What is claimed is:

1. A thickness measurement device for measuring a thickness of a measurement target by using an ultrasonic wave, the thickness measurement device being to be attached to an object including the measurement target therein, the thickness measurement device comprising:
a plurality of ultrasonic elements each configured to transmit the ultrasonic wave to the measurement target from a surface of the object, receive a reflected wave reflected by the measurement target, and output a reception signal; and
a controller configured to control the ultrasonic elements, wherein
the plurality of ultrasonic elements transmit the ultrasonic waves in directions different from one another, and
the controller compares a signal intensity of the reception signal with a predetermined threshold, and measures the thickness of the measurement target based on the reception signal having a signal intensity larger than the threshold.

2. The thickness measurement device according to claim 1, wherein
the plurality of ultrasonic elements transmit the ultrasonic waves in directions away from one another.

3. The thickness measurement device according to claim 2, wherein
the plurality of ultrasonic elements include a plurality of ultrasonic elements disposed along a first axis and a plurality of ultrasonic elements disposed along a second axis orthogonal to the first axis,
with an axis orthogonal to the first axis and the second axis as a third axis, the plurality of ultrasonic elements disposed along the first axis transmit the ultrasonic waves in the directions away from one another in a first plane including the first axis and the third axis, and
the plurality of ultrasonic elements disposed along the second axis transmit the ultrasonic waves in the directions away from one another in a second plane including the second axis and the third axis.
